SEQUENCE LISTING (43 sequences)

SEQ ID NO: 1 - DNA, 8733 bp, Homo sapiens
  gene (1)..(8733): mechanistic target of rapamycin (serine/ threonine kinase) (mTOR)
gctcccggcttagaggacagcggggaaggcgggcggtggggcagggggcctgaagcggcggtaccggtgctggcggcggcagctgaggccttggccgaagccgcgcgaacctcagggcaagatgcttggaaccggacctgccgccgccaccaccgctgccaccacatctagcaatgtgagcgtcctgcagcagtttgccagtggcctaaagagccggaatgaggaaaccagggccaaagccgccaaggagctccagcactatgtcaccatggaactccgagagatgagtcaagaggagtctactcgcttctatgaccaactgaaccatcacatttttgaattggtttccagctcagatgccaatgagaggaaaggtggcatcttggccatagctagcctcataggagtggaaggtgggaatgccacccgaattggcagatttgccaactatcttcggaacctcctcccctccaatgacccagttgtcatggaaatggcatccaaggccattggccgtcttgccatggcaggggacacttttaccgctgagtacgtggaatttgaggtgaagcgagccctggaatggctgggtgctgaccgcaatgagggccggagacatgcagctgtcctggttctccgtgagctggccatcagcgtccctaccttcttcttccagcaagtgcaacccttctttgacaacatttttgtggccgtgtgggaccccaaacaggccatccgtgagggagctgtagccgcccttcgtgcctgtctgattctcacaacccagcgtgagccgaaggagatgcagaagcctcagtggtacaggcacacatttgaagaagcagagaagggatttgatgagaccttggccaaagagaagggcatgaatcgggatgatcggatccatggagccttgttgatccttaacgagctggtccgaatcagcagcatggagggagagcgtctgagagaagaaatggaagaaatcacacagcagcagctggtacacgacaagtactgcaaagatctcatgggcttcggaacaaaacctcgtcacattacccccttcaccagtttccaggctgtacagccccagcagtcaaatgccttggtggggctgctggggtacagctctcaccaaggcctcatgggatttgggacctcccccagtccagctaagtccaccctggtggagagccggtgttgcagagacttgatggaggagaaatttgatcaggtgtgccagtgggtgctgaaatgcaggaatagcaagaactcgctgatccaaatgacaatccttaatttgttgccccgcttggctgcattccgaccttctgccttcacagatacccagtatctccaagataccatgaaccatgtcctaagctgtgtcaagaaggagaaggaacgtacagcggccttccaagccctggggctactttctgtggctgtgaggtctgagtttaaggtctatttgcctcgcgtgctggacatcatccgagcggccctgcccccaaaggacttcgcccataagaggcagaaggcaatgcaggtggatgccacagtcttcacttgcatcagcatgctggctcgagcaatggggccaggcatccagcaggatatcaaggagctgctggagcccatgctggcagtgggactaagccctgccctcactgcagtgctctacgacctgagccgtcagattccacagctaaagaaggacattcaagatgggctactgaaaatgctgtccctggtccttatgcacaaaccccttcgccacccaggcatgcccaagggcctggcccatcagctggcctctcctggcctcacgaccctccctgaggccagcgatgtgggcagcatcactcttgccctccgaacgcttggcagctttgaatttgaaggccactctctgacccaatttgttcgccactgtgcggatcatttcctgaacagtgagcacaaggagatccgcatggaggctgcccgcacctgctcccgcctgctcacaccctccatccacctcatcagtggccatgctcatgtggttagccagaccgcagtgcaagtggtggcagatgtgcttagcaaactgctcgtagttgggataacagatcctgaccctgacattcgctactgtgtcttggcgtccctggacgagcgctttgatgcacacctggcccaggcggagaacttgcaggccttgtttgtggctctgaatgaccaggtgtttgagatccgggagctggccatctgcactgtgggccgactcagtagcatgaaccctgcctttgtcatgcctttcctgcgcaagatgctcatccagattttgacagagttggagcacagtgggattggaagaatcaaagagcagagtgcccgcatgctggggcacctggtctccaatgccccccgactcatccgcccctacatggagcctattctgaaggcattaattttgaaactgaaagatccagaccctgatccaaacccaggtgtgatcaataatgtcctggcaacaataggagaattggcacaggttagtggcctggaaatgaggaaatgggttgatgaactttttattatcatcatggacatgctccaggattcctctttgttggccaaaaggcaggtggctctgtggaccctgggacagttggtggccagcactggctatgtagtagagccctacaggaagtaccctactttgcttgaggtgctactgaattttctgaagactgagcagaaccagggtacacgcagagaggccatccgtgtgttagggcttttaggggctttggatccttacaagcacaaagtgaacattggcatgatagaccagtcccgggatgcctctgctgtcagcctgtcagaatccaagtcaagtcaggattcctctgactatagcactagtgaaatgctggtcaacatgggaaacttgcctctggatgagttctacccagctgtgtccatggtggccctgatgcggatcttccgagaccagtcactctctcatcatcacaccatggttgtccaggccatcaccttcatcttcaagtccctgggactcaaatgtgtgcagttcctgccccaggtcatgcccacgttccttaacgtcattcgagtctgtgatggggccatccgggaatttttgttccagcagctgggaatgttggtgtcctttgtgaagagccacatcagaccttatatggatgaaatagtcaccctcatgagagaattctgggtcatgaacacctcaattcagagcacgatcattcttctcattgagcaaattgtggtagctcttgggggtgaatttaagctctacctgccccagctgatcccacacatgctgcgtgtcttcatgcatgacaacagcccaggccgcattgtctctatcaagttactggctgcaatccagctgtttggcgccaacctggatgactacctgcatttactgctgcctcctattgttaagttgtttgatgcccctgaagctccactgccatctcgaaaggcagcgctagagactgtggaccgcctgacggagtccctggatttcactgactatgcctcccggatcattcaccctattgttcgaacactggaccagagcccagaactgcgctccacagccatggacacgctgtcttcacttgtttttcagctggggaagaagtaccaaattttcattccaatggtgaataaagttctggtgcgacaccgaatcaatcatcagcgctatgatgtgctcatctgcagaattgtcaagggatacacacttgctgatgaagaggaggatcctttgatttaccagcatcggatgcttaggagtggccaaggggatgcattggctagtggaccagtggaaacaggacccatgaagaaactgcacgtcagcaccatcaacctccaaaaggcctggggcgctgccaggagggtctccaaagatgactggctggaatggctgagacggctgagcctggagctgctgaaggactcatcatcgccctccctgcgctcctgctgggccctggcacaggcctacaacccgatggccagggatctcttcaatgctgcatttgtgtcctgctggtctgaactgaatgaagatcaacaggatgagctcatcagaagcatcgagttggccctcacctcacaagacatcgctgaagtcacacagaccctcttaaacttggctgaattcatggaacacagtgacaagggccccctgccactgagagatgacaatggcattgttctgctgggtgagagagctgccaagtgccgagcatatgccaaagcactacactacaaagaactggagttccagaaaggccccacccctgccattctagaatctctcatcagcattaataataagctacagcagccggaggcagcggccggagtgttagaatatgccatgaaacactttggagagctggagatccaggctacctggtatgagaaactgcacgagtgggaggatgcccttgtggcctatgacaagaaaatggacaccaacaaggacgacccagagctgatgctgggccgcatgcgctgcctcgaggccttgggggaatggggtcaactccaccagcagtgctgtgaaaagtggaccctggttaatgatgagacccaagccaagatggcccggatggctgctgcagctgcatggggtttaggtcagtgggacagcatggaagaatacacctgtatgatccctcgggacacccatgatggggcattttatagagctgtgctggcactgcatcaggacctcttctccttggcacaacagtgcattgacaaggccagggacctgctggatgctgaattaactgcgatggcaggagagagttacagtcgggcatatggggccatggtttcttgccacatgctgtccgagctggaggaggttatccagtacaaacttgtccccgagcgacgagagatcatccgccagatctggtgggagagactgcagggctgccagcgtatcgtagaggactggcagaaaatccttatggtgcggtcccttgtggtcagccctcatgaagacatgagaacctggctcaagtatgcaagcctgtgcggcaagagtggcaggctggctcttgctcataaaactttagtgttgctcctgggagttgatccgtctcggcaacttgaccatcctctgccaacagttcaccctcaggtgacctatgcctacatgaaaaacatgtggaagagtgcccgcaagatcgatgccttccagcacatgcagcattttgtccagaccatgcagcaacaggcccagcatgccatcgctactgaggaccagcagcataagcaggaactgcacaagctcatggcccgatgcttcctgaaacttggagagtggcagctgaatctacagggcatcaatgagagcacaatccccaaagtgctgcagtactacagcgccgccacagagcacgaccgcagctggtacaaggcctggcatgcgtgggcagtgatgaacttcgaagctgtgctacactacaaacatcagaaccaagcccgcgatgagaagaagaaactgcgtcatgccagcggggccaacatcaccaacgccaccactgccgccaccacggccgccactgccaccaccactgccagcaccgagggcagcaacagtgagagcgaggccgagagcaccgagaacagccccaccccatcgccgctgcagaagaaggtcactgaggatctgtccaaaaccctcctgatgtacacggtgcctgccgtccagggcttcttccgttccatctccttgtcacgaggcaacaacctccaggatacactcagagttctcaccttatggtttgattatggtcactggccagatgtcaatgaggccttagtggagggggtgaaagccatccagattgatacctggctacaggttatacctcagctcattgcaagaattgatacgcccagacccttggtgggacgtctcattcaccagcttctcacagacattggtcggtaccacccccaggccctcatctacccactgacagtggcttctaagtctaccacgacagcccggcacaatgcagccaacaagattctgaagaacatgtgtgagcacagcaacaccctggtccagcaggccatgatggtgagcgaggagctgatccgagtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagcagctgcctcagctcacatccttagagctgcaatatgtttccccaaaacttctgatgtgccgggaccttgaattggctgtgccaggaacatatgaccccaaccagccaatcattcgcattcagtccatagcaccgtctttgcaagtcatcacatccaagcagaggccccggaaattgacacttatgggcagcaacggacatgagtttgttttccttctaaaaggccatgaagatctgcgccaggatgagcgtgtgatgcagctcttcggcctggttaacacccttctggccaatgacccaacatctcttcggaaaaacctcagcatccagagatacgctgtcatccctttatcgaccaactcgggcctcattggctgggttccccactgtgacacactgcacgccctcatccgggactacagggagaagaagaagatccttctcaacatcgagcatcgcatcatgttgcggatggctccggactatgaccacttgactctgatgcagaaggtggaggtgtttgagcatgccgtcaataatacagctggggacgacctggccaagctgctgtggctgaaaagccccagctccgaggtgtggtttgaccgaagaaccaattatacccgttctttagcggtcatgtcaatggttgggtatattttaggcctgggagatagacacccatccaacctgatgctggaccgtctgagtgggaagatcctgcacattgactttggggactgctttgaggttgctatgacccgagagaagtttccagagaagattccatttagactaacaagaatgttgaccaatgctatggaggttacaggcctggatggcaactacagaatcacatgccacacagtgatggaggtgctgcgagagcacaaggacagtgtcatggccgtgctggaagcctttgtctatgaccccttgctgaactggaggctgatggacacaaataccaaaggcaacaagcgatcccgaacgaggacggattcctactctgctggccagtcagtcgaaattttggacggtgtggaacttggagagccagcccataagaaaacggggaccacagtgccagaatctattcattctttcattggagacggtttggtgaaaccagaggccctaaataagaaagctatccagattattaacagggttcgagataagctcactggtcgggacttctctcatgatgacactttggatgttccaacgcaagttgagctgctcatcaaacaagcgacatcccatgaaaacctctgccagtgctatattggctggtgccctttctggtaactggaggcccagatgtgcccatcacgttttttctgaggcttttgtactttagtaaatgcttccactaaactgaaaccatggtgagaaagtttgactttgttaaatattttgaaatgtaaatgaaaagaactactgtatattaaaagttggtttgaaccaactttctagctgctgttgaagaatatattgtcagaaacacaaggcttgatttggttcccaggacagtgaaacatagtaataccacgtaaatcaagccattcattttggggaacagaagatccataactttagaaatacgggttttgacttaactcacaagagaactcatcataagtacttgctgatggaagaatgacctagttgctcctctcaacatgggtacagcaaactcagcacagccaagaagcctcaggtcgtggagaacatggattaggatcctagactgtaaagacacagaagatgctgacctcacccctgccacctatcccaagacctcactggtctgtggacagcagcagaaatgtttgcaagataggccaaaatgagtacaaaaggtctgtcttccatcagacccagtgatgctgcgactcacacgcttcaattcaagacctgaccgctagtagggaggtttattcagatcgctggcagcctcggctgagcagatgcacagaggggatcactgtgcagtgggaccaccctcactggccttctgcagcagggttctgggatgttttcagtggtcaaaatactctgtttagagcaagggctcagaaaacagaaatactgtcatggaggtgctgaacacagggaaggtctggtacatattggaaattatgagcagaacaaatactcaactaaatgcacaaagtataaagtgtagccatgtctagacaccatgttgtatcagaataatttttgtgccaataaatgacatcagaattttaaacatatgtaaaaaaaaa

SEQ ID NO: 2 - DNA, 6055 bp, Homo sapiens
  gene (1)..(6055): kinase insert domain receptor (a type III receptor tyrosine kinase)
actgagtcccgggaccccgggagagcggtcaatgtgtggtcgctgcgtttcctctgcctgcgccgggcatcacttgcgcgccgcagaaagtccgtctggcagcctggatatcctctcctaccggcacccgcagacgcccctgcagccgcggtcggcgcccgggctccctagccctgtgcgctcaactgtcctgcgctgcggggtgccgcgagttccacctccgcgcctccttctctagacaggcgctgggagaaagaaccggctcccgagttctgggcatttcgcccggctcgaggtgcaggatgcagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaaatcattattctagtaggcacggcggtgattgccatgttcttctggctacttcttgtcatcatcctacggaccgttaagcgggccaatggaggggaactgaagacaggctacttgtccatcgtcatggatccagatgaactcccattggatgaacattgtgaacgactgccttatgatgccagcaaatgggaattccccagagaccggctgaagctaggtaagcctcttggccgtggtgcctttggccaagtgattgaagcagatgcctttggaattgacaagacagcaacttgcaggacagtagcagtcaaaatgttgaaagaaggagcaacacacagtgagcatcgagctctcatgtctgaactcaagatcctcattcatattggtcaccatctcaatgtggtcaaccttctaggtgcctgtaccaagccaggagggccactcatggtgattgtggaattctgcaaatttggaaacctgtccacttacctgaggagcaagagaaatgaatttgtcccctacaagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaagctcctgaagatctgtataaggacttcctgaccttggagcatctcatctgttacagcttccaagtggctaagggcatggagttcttggcatcgcgaaagtgtatccacagggacctggcggcacgaaatatcctcttatcggagaagaacgtggttaaaatctgtgactttggcttggcccgggatatttataaagatccagattatgtcagaaaaggagatgctcgcctccctttgaaatggatggccccagaaacaatttttgacagagtgtacacaatccagagtgacgtctggtcttttggtgttttgctgtgggaaatattttccttaggtgcttctccatatcctggggtaaagattgatgaagaattttgtaggcgattgaaagaaggaactagaatgagggcccctgattatactacaccagaaatgtaccagaccatgctggactgctggcacggggagcccagtcagagacccacgttttcagagttggtggaacatttgggaaatctcttgcaagctaatgctcagcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcagtcagtatctgcagaacagtaagcgaaagagccggcctgtgagtgtaaaaacatttgaagatatcccgttagaagaaccagaagtaaaagtaatcccagatgacaaccagacggacagtggtatggttcttgcctcagaagagctgaaaactttggaagacagaaccaaattatctccatcttttggtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgactcggggaccacactgagctctcctcctgtttaaaaggaagcatccacacccccaactcctggacatcacatgagaggtgctgctcagattttcaagtgttgttctttccaccagcaggaagtagccgcatttgattttcatttcgacaacagaaaaaggacctcggactgcagggagccagtcttctaggcatatcctggaagaggcttgtgacccaagaatgtgtctgtgtcttctcccagtgttgacctgatcctctttttcattcatttaaaaagcatttatcatgccccctgctgcgggtctcaccatgggtttagaacaaagacgttcaagaaatggccccatcctcaaagaagtagcagtacctggggagctgacacttctgtaaaactagaagataaaccaggcaatgtaagtgttcgaggtgttgaagatgggaaggatttgcagggctgagtctatccaagaggctttgtttaggacgtgggtcccaagccaagccttaagtgtggaattcggattgatagaaaggaagactaacgttaccttgctttggagagtactggagcctgcaaatgcattgtgtttgctctggtggaggtgggcatggggtctgttctgaaatgtaaagggttcagacggggtttctggttttagaaggttgcgtgttcttcgagttgggctaaagtagagttcgttgtgctgtttctgactcctaatgagagttccttccagaccgttacgtgtctcctggccaagccccaggaaggaaatgatgcagctctggctccttgtctcccaggctgatcctttattcagaataccacaaagaaaggacattcagctcaaggctccctgccgtgttgaagagttctgactgcacaaaccagcttctggtttcttctggaatgaataccctcatatctgtcctgatgtgatatgtctgagactgaatgcgggaggttcaatgtgaagctgtgtgtggtgtcaaagtttcaggaaggattttacccttttgttcttccccctgtccccaacccactctcaccccgcaacccatcagtattttagttatttggcctctactccagtaaacctgattgggtttgttcactctctgaatgattattagccagacttcaaaattattttatagcccaaattataacatctattgtattatttagacttttaacatatagagctatttctactgatttttgcccttgttctgtcctttttttcaaaaaagaaaatgtgttttttgtttggtaccatagtgtgaaatgctgggaacaatgactataagacatgctatggcacatatatttatagtctgtttatgtagaaacaaatgtaatatattaaagccttatatataatgaactttgtactattcacattttgtatcagtattatgtagcataacaaaggtcataatgctttcagcaattgatgtcattttattaaagaacattgaaaaacttgaaggaatccctttgcaaggttgcattactgtacccatcatttctaaaatggaagagggggtggctgggcacagtggccgacacctaaaaacccagcactttggggggccaaggtgggaggatcgcttgagcccaggagttcaagaccagtctggccaacatggtcagattccatctcaaagaaaaaaggtaaaaataaaataaaatggagaagaaggaatcaga

SEQ ID NO: 3 - DNA, 5718 bp, Homo sapiens
  gene (1)..(5718): platelet-derived growth factor receptor, beta polypeptide (PDGFRB)
ctcctgaggctgccagcagccagcagtgactgcccgccctatctgggacccaggatcgctctgtgagcaacttggagccagagaggagatcaacaaggaggaggagagagccggcccctcagccctgctgcccagcagcagcctgtgctcgccctgcccaacgcagacagccagacccagggcggcccctctggcggctctgctcctcccgaaggatgcttggggagtgaggcgaagctgggccgctcctctcccctacagcagcccccttcctccatccctctgttctcctgagccttcaggagcctgcaccagtcctgcctgtccttctactcagctgttacccactctgggaccagcagtctttctgataactgggagagggcagtaaggaggacttcctggagggggtgactgtccagagcctggaactgtgcccacaccagaagccatcagcagcaaggacaccatgcggcttccgggtgcgatgccagctctggccctcaaaggcgagctgctgttgctgtctctcctgttacttctggaaccacagatctctcagggcctggtcgtcacacccccggggccagagcttgtcctcaatgtctccagcaccttcgttctgacctgctcgggttcagctccggtggtgtgggaacggatgtcccaggagcccccacaggaaatggccaaggcccaggatggcaccttctccagcgtgctcacactgaccaacctcactgggctagacacgggagaatacttttgcacccacaatgactcccgtggactggagaccgatgagcggaaacggctctacatctttgtgccagatcccaccgtgggcttcctccctaatgatgccgaggaactattcatctttctcacggaaataactgagatcaccattccatgccgagtaacagacccacagctggtggtgacactgcacgagaagaaaggggacgttgcactgcctgtcccctatgatcaccaacgtggcttttctggtatctttgaggacagaagctacatctgcaaaaccaccattggggacagggaggtggattctgatgcctactatgtctacagactccaggtgtcatccatcaacgtctctgtgaacgcagtgcagactgtggtccgccagggtgagaacatcaccctcatgtgcattgtgatcgggaatgaggtggtcaacttcgagtggacatacccccgcaaagaaagtgggcggctggtggagccggtgactgacttcctcttggatatgccttaccacatccgctccatcctgcacatccccagtgccgagttagaagactcggggacctacacctgcaatgtgacggagagtgtgaatgaccatcaggatgaaaaggccatcaacatcaccgtggttgagagcggctacgtgcggctcctgggagaggtgggcacactacaatttgctgagctgcatcggagccggacactgcaggtagtgttcgaggcctacccaccgcccactgtcctgtggttcaaagacaaccgcaccctgggcgactccagcgctggcgaaatcgccctgtccacgcgcaacgtgtcggagacccggtatgtgtcagagctgacactggttcgcgtgaaggtggcagaggctggccactacaccatgcgggccttccatgaggatgctgaggtccagctctccttccagctacagatcaatgtccctgtccgagtgctggagctaagtgagagccaccctgacagtggggaacagacagtccgctgtcgtggccggggcatgccccagccgaacatcatctggtctgcctgcagagacctcaaaaggtgtccacgtgagctgccgcccacgctgctggggaacagttccgaagaggagagccagctggagactaacgtgacgtactgggaggaggagcaggagtttgaggtggtgagcacactgcgtctgcagcacgtggatcggccactgtcggtgcgctgcacgctgcgcaacgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttacgagatccgatggaaggtgattgagtctgtgagctctgacggccatgagtacatctacgtggaccccatgcagctgccctatgactccacgtgggagctgccgcgggaccagcttgtgctgggacgcaccctcggctctggggcctttgggcaggtggtggaggccacggctcatggcctgagccattctcaggccacgatgaaagtggccgtcaagatgcttaaatccacagcccgcagcagtgagaagcaagcccttatgtcggagctgaagatcatgagtcaccttgggccccacctgaacgtggtcaacctgttgggggcctgcaccaaaggaggacccatctatatcatcactgagtactgccgctacggagacctggtggactacctgcaccgcaacaaacacaccttcctgcagcaccactccgacaagcgccgcccgcccagcgcggagctctacagcaatgctctgcccgttgggctccccctgcccagccatgtgtccttgaccggggagagcgacggtggctacatggacatgagcaaggacgagtcggtggactatgtgcccatgctggacatgaaaggagacgtcaaatatgcagacatcgagtcctccaactacatggccccttacgataactacgttccctctgcccctgagaggacctgccgagcaactttgatcaacgagtctccagtgctaagctacatggacctcgtgggcttcagctaccaggtggccaatggcatggagtttctggcctccaagaactgcgtccacagagacctggcggctaggaacgtgctcatctgtgaaggcaagctggtcaagatctgtgactttggcctggctcgagacatcatgcgggactcgaattacatctccaaaggcagcacctttttgcctttaaagtggatggctccggagagcatcttcaacagcctctacaccaccctgagcgacgtgtggtccttcgggatcctgctctgggagatcttcaccttgggtggcaccccttacccagagctgcccatgaacgagcagttctacaatgccatcaaacggggttaccgcatggcccagcctgcccatgcctccgacgagatctatgagatcatgcagaagtgctgggaagagaagtttgagattcggccccccttctcccagctggtgctgcttctcgagagactgttgggcgaaggttacaaaaagaagtaccagcaggtggatgaggagtttctgaggagtgaccacccagccatccttcggtcccaggcccgcttgcctgggttccatggcctccgatctcccctggacaccagctccgtcctctatactgccgtgcagcccaatgagggtgacaacgactatatcatccccctgcctgaccccaaacccgaggttgctgacgagggcccactggagggttcccccagcctagccagctccaccctgaatgaagtcaacacctcctcaaccatctcctgtgacagccccctggagccccaggacgaaccagagccagagccccagcttgagctccaggtggagccggagccagagctggaacagttgccggattcggggtgccctgcgcctcgggcggaagcagaggatagcttcctgtagggggctggcccctaccctgccctgcctgaagctccccccctgccagcacccagcatctcctggcctggcctgaccgggcttcctgtcagccaggctgcccttatcagctgtccccttctggaagctttctgctcctgacgtgttgtgccccaaaccctggggctggcttaggaggcaagaaaactgcaggggccgtgaccagccctctgcctccagggaggccaactgactctgagccagggttcccccagggaactcagttttcccatatgtaagatgggaaagttaggcttgatgacccagaatctaggattctctccctggctgacaggtggggagaccgaatccctccctgggaagattcttggagttactgaggtggtaaattaacttttttctgttcagccagctacccctcaaggaatcatagctctctcctcgcacttttatccacccaggagctagggaagagaccctagcctccctggctgctggctgagctagggcctagccttgagcagtgttgcctcatccagaagaaagccagtctcctccctatgatgccagtccctgcgttccctggcccgagctggtctggggccattaggcagcctaattaatgctggaggctgagccaagtacaggacacccccagcctgcagcccttgcccagggcacttggagcacacgcagccatagcaagtgcctgtgtccctgtccttcaggcccatcagtcctggggctttttctttatcaccctcagtcttaatccatccaccagagtctagaaggccagacgggccccgcatctgtgatgagaatgtaaatgtgccagtgtggagtggccacgtgtgtgtgccagtatatggccctggctctgcattggacctgctatgaggctttggaggaatccctcaccctctctgggcctcagtttccccttcaaaaaatgaataagtcggacttattaactctgagtgccttgccagcactaacattctagagtattccaggtggttgcacatttgtccagatgaagcaaggccatataccctaaacttccatcctgggggtcagctgggctcctgggagattccagatcacacatcacactctggggactcaggaaccatgccccttccccaggcccccagcaagtctcaagaacacagctgcacaggccttgacttagagtgacagccggtgtcctggaaagcccccagcagctgccccagggacatgggaagaccacgggacctctttcactacccacgatgacctccgggggtatcctgggcaaaagggacaaagagggcaaatgagatcacctcctgcagcccaccactccagcacctgtgccgaggtctgcgtcgaagacagaatggacagtgaggacagttatgtcttgtaaaagacaagaagcttcagatgggtaccccaagaaggatgtgagaggtgggcgctttggaggtttgcccctcacccaccagctgccccatccctgaggcagcgctccatgggggtatggttttgtcactgcccagacctagcagtgacatctcattgtccccagcccagtgggcattggaggtgccaggggagtcagggttgtagccaagacgcccccgcacggggagggttgggaagggggtgcaggaagctcaacccctctgggcaccaaccctgcattgcaggttggcaccttacttccctgggatccccagagttggtccaaggagggagagtgggttctcaatacggtaccaaagatataatcacctaggtttacaaatatttttaggactcacgttaactcacatttatacagcagaaatgctattttgtatgctgttaagtttttctatctgtgtacttttttttaagggaaagattttaatattaaacctggtgcttctcactcacaaaaa

SEQ ID NO: 4 - DNA, 5917 bp, Homo sapiens
  gene (1)..(5917): fibroblast growth factor receptor 1 (FGFR1)
agatgcaggggcgcaaacgccaaaggagaccaggctgtaggaagagaagggcagagcgccggacagctcggcccgctccccgtcctttggggccgcggctggggaactacaaggcccagcaggcagctgcagggggcggaggcggaggagggaccagcgcgggtgggagtgagagagcgagccctcgcgccccgccggcgcatagcgctcggagcgctcttgcggccacaggcgcggcgtcctcggcggcgggcggcagctagcgggagccgggacgccggtgcagccgcagcgcgcggaggaacccgggtgtgccgggagctgggcggccacgtccggacgggaccgagacccctcgtagcgcattgcggcgacctcgccttccccggccgcgagcgcgccgctgcttgaaaagccgcggaacccaaggacttttctccggtccgagctcggggcgccccgcagggcgcacggtacccgtgctgcagtcgggcacgccgcggcgccggggcctccgcagggcgatggagcccggtctgcaaggaaagtgaggcgccgccgctgcgttctggaggaggggggcacaaggtctggagaccccgggtggcggacgggagccctccccccgccccgcctccggggcaccagctccggctccattgttcccgcccgggctggaggcgccgagcaccgagcgccgccgggagtcgagcgccggccgcggagctcttgcgaccccgccaggacccgaacagagcccgggggcggcgggccggagccggggacgcgggcacacgcccgctcgcacaagccacggcggactctcccgaggcggaacctccacgccgagcgagggtcagtttgaaaaggaggatcgagctcactgtggagtatccatggagatgtggagccttgtcaccaacctctaactgcagaactgggatgtggagctggaagtgcctcctcttctgggctgtgctggtcacagccacactctgcaccgctaggccgtccccgaccttgcctgaacaagcccagccctggggagcccctgtggaagtggagtccttcctggtccaccccggtgacctgctgcagcttcgctgtcggctgcgggacgatgtgcagagcatcaactggctgcgggacggggtgcagctggcggaaagcaaccgcacccgcatcacaggggaggaggtggaggtgcaggactccgtgcccgcagactccggcctctatgcttgcgtaaccagcagcccctcgggcagtgacaccacctacttctccgtcaatgtttcagatgctctcccctcctcggaggatgatgatgatgatgatgactcctcttcagaggagaaagaaacagataacaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctgactgccacccacacgccctccccagactccaccgtcagctgtaaccctcacccacagcccctgctgggcccaccacctgtccgtccctgtcccctttcctgctggcaggagccggctgcctaccaggggccttcctgtgtggcctgccttcaccccactcagctcacctctccctccacctcctctccacctgctggtgagaggtgcaaagaggcagatctttgctgccagccacttcatcccctcccagatgttggaccaacacccctccctgccaccaggcactgcctggagggcagggagtgggagccaatgaacaggcatgcaagtgagagcttcctgagctttctcctgtcggtttggtctgttttgccttcacccataagcccctcgcactctggtggcaggtgccttgtcctcagggctacagcagtagggaggtcagtgcttcgtgcctcgattgaaggtgacctctgccccagataggtggtgccagtggcttattaattccgatactagtttgctttgctgaccaaatgcctggtaccagaggatggtgaggcgaaggccaggttgggggcagtgttgtggccctggggcccagccccaaactgggggctctgtatatagctatgaagaaaacacaaagtgtataaatctgagtatatatttacatgtctttttaaaagggtcgttaccagagatttacccatcgggtaagatgctcctggtggctgggaggcatcagttgctatatattaaaaacaaaaaagaaaaaaaaggaaaatgtttttaaaaaggtcatatattttttgctacttttgctgttttatttttttaaattatgttctaaacctattttcagtttaggtccctcaataaaaattgctgctgcttcatttatctatgggctgtatgaaaagggtgggaatgtccactggaaagaagggacacccacgggccctggggctaggtctgtcccgagggcaccgcatgctcccggcgcaggttccttgtaacctcttcttcctaggtcctgcacccagacctcacgacgcacctcctgcctctccgctgcttttggaaagtcagaaaaagaagatgtctgcttcgagggcaggaaccccatccatgcagtagaggcgctgggcagagagtcaaggcccagcagccatcgaccatggatggtttcctccaaggaaaccggtggggttgggctggggagggggcacctacctaggaatagccacggggtagagctacagtgattaagaggaaagcaagggcgcggttgctcacgcctgtaatcccagcactttgggacaccgaggtgggcagatcacttcaggtcaggagtttgagaccagcctggccaacttagtgaaaccccatctctactaaaaatgcaaaaattatccaggcatggtggcacacgcctgtaatcccagctccacaggaggctgaggcagaatcccttgaagctgggaggcggaggttgcagtgagccgagattgcgccattgcactccagcctgggcaacagagaaaacaaaaaggaaaacaaatgatgaaggtctgcagaaactgaaacccagacatgtgtctgccccctctatgtgggcatggttttgccagtgcttctaagtgcaggagaacatgtcacctgaggctagttttgcattcaggtccctggcttcgtttcttgttggtatgcctccccagatcgtccttcctgtatccatgtgaccagactgtatttgttgggactgtcgcagatcttggcttcttacagttcttcctgtccaaactccatcctgtccctcaggaacggggggaaaattctccgaatgtttttggttttttggctgcttggaatttacttctgccacctgctggtcatcactgtcctcactaagtggattctggctcccccgtacctcatggctcaaactaccactcctcagtcgctatattaaagcttatattttgctggattactgctaaatacaaaagaaagttcaatatgttttcatttctgtagggaaaatgggattgctgctttaaatttctgagctagggattttttggcagctgcagtgttggcgactattgtaaaattctctttgtttctctctgtaaatagcacctgctaacattacaatttgtatttatgtttaaagaaggcatcatttggtgaacagaactaggaaatgaatttttagctcttaaaagcatttgctttgagaccgcacaggagtgtctttccttgtaaaacagtgatgataatttctgccttggccctaccttgaagcaatgttgtgtgaagggatgaagaatctaaaagtcttcataagtccttgggagaggtgctagaaaaatataaggcactatcataattacagtgatgtccttgctgttactactcaaatcacccacaaatttccccaaagactgcgctagctgtcaaataaaagacagtgaaattgacctgaaaaaaaaaaaaaaaaa

SEQ ID NO: 5 - DNA, 5190 bp, Homo sapiens
  gene (1)..(5190): v-kit Hardy-Zuckerman 4 feline sarcoma viral oncogene homolog (KIT)
tctgggggctcggctttgccgcgctcgctgcacttgggcgagagctggaacgtggaccagagctcggatcccatcgcagctaccgcgatgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctgagcagaatcagtgtttgggtcacccctccaggaatgatctcttcttttggcttccatgatggttattttcttttctttcaacttgcatccaactccaggatagtgggcaccccactgcaatcctgtctttctgagcacactttagtggccgatgatttttgtcatcagccaccatcctattgcaaaggttccaactgtatatattcccaatagcaacgtagcttctaccatgaacagaaaacattctgatttggaaaaagagagggaggtatggactgggggccagagtcctttccaaggcttctccaattctgcccaaaaatatggttgatagtttacctgaataaatggtagtaatcacagttggccttcagaaccatccatagtagtatgatgatacaagattagaagctgaaaacctaagtcctttatgtggaaaacagaacatcattagaacaaaggacagagtatgaacacctgggcttaagaaatctagtatttcatgctgggaatgagacataggccatgaaaaaaatgatccccaagtgtgaacaaaagatgctcttctgtggaccactgcatgagcttttatactaccgacctggtttttaaatagagtttgctattagagcattgaattggagagaaggcctccctagccagcacttgtatatacgcatctataaattgtccgtgttcatacatttgaggggaaaacaccataaggtttcgtttctgtatacaaccctggcattatgtccactgtgtatagaagtagattaagagccatataagtttgaaggaaacagttaataccattttttaaggaaacaatataaccacaaagcacagtttgaacaaaatctcctcttttagctgatgaacttattctgtagattctgtggaacaagcctatcagcttcagaatggcattgtactcaatggatttgatgctgtttgacaaagttactgattcactgcatggctcccacaggagtgggaaaacactgccatcttagtttggattcttatgtagcaggaaataaagtataggtttagcctccttcgcaggcatgtcctggacaccgggccagtatctatatatgtgtatgtacgtttgtatgtgtgtagacaaatatttggaggggtatttttgccctgagtccaagagggtcctttagtacctgaaaagtaacttggctttcattattagtactgctcttgtttcttttcacatagctgtctagagtagcttaccagaagcttccatagtggtgcagaggaagtggaaggcatcagtccctatgtatttgcagttcacctgcacttaaggcactctgttatttagactcatcttactgtacctgttccttagaccttccataatgctactgtctcactgaaacatttaaattttaccctttagactgtagcctggatattattcttgtagtttacctctttaaaaacaaaacaaaacaaaacaaaaaactccccttcctcactgcccaatataaaaggcaaatgtgtacatggcagagtttgtgtgttgtcttgaaagattcaggtatgttgcctttatggtttcccccttctacatttcttagactacatttagagaactgtggccgttatctggaagtaaccatttgcactggagttctatgctctcgcacctttccaaagttaacagattttggggttgtgttgtcacccaagagattgttgtttgccatactttgtctgaaaaattcctttgtgtttctattgacttcaatgatagtaagaaaagtggttgttagttatagatgtctaggtacttcaggggcacttcattgagagttttgtcttggatattcttgaaagtttatatttttataattttttcttacatcagatgtttctttgcagtggcttaatgtttgaaattattttgtggctttttttgtaaatattgaaatgtagcaataatgtcttttgaatattcccaagcccatgagtccttgaaaatattttttatatatacagtaactttatgtgtaaatacataagcggcgtaagtttaaaggatgttggtgttccacgtgttttattcctgtatgttgtccaattgttgacagttctgaagaattctaataaaatgtacatatataaatcaaaaaaaaaaaaaaaa

SEQ ID NO: 6 - DNA, 5616 bp, Homo sapiens
  gene (1)..(5616): epidermal growth factor receptor (EGFR)
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 7 - DNA, 3291 bp, Homo sapiens
  gene (1)..(3291): cRAF
agaatcggagagccggtggcgtcgcaggtcgggaggacgagcaccgagtcgagggctcgctcgtctgggccgcccgagagtcttaatcgcgggcgcttgggccgccatcttagatggcgggagtaagaggaaaacgattgtgaggcgggaacggctttctgctgccttttttgggccccgaaaagggtcagctggccgggctttggggcgcgtgccctgaggcgcggagcgcgtttgctacgatgcgggggctgctcggggctccgtcccctgggctggggacgcgccgaatgtgaccgcctcccgctccctcacccgccgcggggaggaggagcgggcgagaagctgccgccgaacgacaggacgttggggcggcctggctccctcaggtttaagaattgtttaagctgcatcaatggagcacatacagggagcttggaagacgatcagcaatggttttggattcaaagatgccgtgtttgatggctccagctgcatctctcctacaatagttcagcagtttggctatcagcgccgggcatcagatgatggcaaactcacagatccttctaagacaagcaacactatccgtgttttcttgccgaacaagcaaagaacagtggtcaatgtgcgaaatggaatgagcttgcatgactgccttatgaaagcactcaaggtgaggggcctgcaaccagagtgctgtgcagtgttcagacttctccacgaacacaaaggtaaaaaagcacgcttagattggaatactgatgctgcgtctttgattggagaagaacttcaagtagatttcctggatcatgttcccctcacaacacacaactttgctcggaagacgttcctgaagcttgccttctgtgacatctgtcagaaattcctgctcaatggatttcgatgtcagacttgtggctacaaatttcatgagcactgtagcaccaaagtacctactatgtgtgtggactggagtaacatcagacaactcttattgtttccaaattccactattggtgatagtggagtcccagcactaccttctttgactatgcgtcgtatgcgagagtctgtttccaggatgcctgttagttctcagcacagatattctacacctcacgccttcacctttaacacctccagtccctcatctgaaggttccctctcccagaggcagaggtcgacatccacacctaatgtccacatggtcagcaccaccctgcctgtggacagcaggatgattgaggatgcaattcgaagtcacagcgaatcagcctcaccttcagccctgtccagtagccccaacaatctgagcccaacaggctggtcacagccgaaaacccccgtgccagcacaaagagagcgggcaccagtatctgggacccaggagaaaaacaaaattaggcctcgtggacagagagattcaagctattattgggaaatagaagccagtgaagtgatgctgtccactcggattgggtcaggctcttttggaactgtttataagggtaaatggcacggagatgttgcagtaaagatcctaaaggttgtcgacccaaccccagagcaattccaggccttcaggaatgaggtggctgttctgcgcaaaacacggcatgtgaacattctgcttttcatggggtacatgacaaaggacaacctggcaattgtgacccagtggtgcgagggcagcagcctctacaaacacctgcatgtccaggagaccaagtttcagatgttccagctaattgacattgcccggcagacggctcagggaatggactatttgcatgcaaagaacatcatccatagagacatgaaatccaacaatatatttctccatgaaggcttaacagtgaaaattggagattttggtttggcaacagtaaagtcacgctggagtggttctcagcaggttgaacaacctactggctctgtcctctggatggccccagaggtgatccgaatgcaggataacaacccattcagtttccagtcggatgtctactcctatggcatcgtattgtatgaactgatgacgggggagcttccttattctcacatcaacaaccgagatcagatcatcttcatggtgggccgaggatatgcctccccagatcttagtaagctatataagaactgccccaaagcaatgaagaggctggtagctgactgtgtgaagaaagtaaaggaagagaggcctctttttccccagatcctgtcttccattgagctgctccaacactctctaccgaagatcaaccggagcgcttccgagccatccttgcatcgggcagcccacactgaggatatcaatgcttgcacgctgaccacgtccccgaggctgcctgtcttctagttgactttgcacctgtcttcaggctgccaggggaggaggagaagccagcaggcaccacttttctgctccctttctccagaggcagaacacatgttttcagagaagctgctgctaaggaccttctagactgctcacagggccttaacttcatgttgccttcttttctatccctttgggccctgggagaaggaagccatttgcagtgctggtgtgtcctgctccctccccacattccccatgctcaaggcccagccttctgtagatgcgcaagtggatgttgatggtagtacaaaaagcaggggcccagccccagctgttggctacatgagtatttagaggaagtaaggtagcaggcagtccagccctgatgtggagacacatgggattttggaaatcagcttctggaggaatgcatgtcacaggcgggactttcttcagagagtggtgcagcgccagacattttgcacataaggcaccaaacagcccaggactgccgagactctggccgcccgaaggagcctgctttggtactatggaacttttcttaggggacacgtcctcctttcacagcttctaaggtgtccagtgcattgggatggttttccaggcaaggcactcggccaatccgcatctcagccctctcagggagcagtcttccatcatgctgaattttgtcttccaggagctgcccctatggggcggggccgcagggccagccttgtttctctaacaaacaaacaaacaaacagccttgtttctctagtcacatcatgtgtatacaaggaagccaggaatacaggttttcttgatgatttgggttttaattttgtttttattgcacctgacaaaatacagttatctgatggtccctcaattatgttattttaataaaataaattaaatttaggtgtaaaaaaaaaaaaaaaaaa

SEQ ID NO: 8 - DNA, 23 bp, Artificial Sequence
  VEGFR2 forward primer
caccactcaaacgctgacatgta

SEQ ID NO: 9 - DNA, 22 bp, Artificial Sequence
  VEGFR2 reverse primer
ccaactgccaataccagtggat

SEQ ID NO: 10 - DNA, 23 bp, Artificial Sequence
  VEGFR2 probe
tatgccattcctcccccgcatca

SEQ ID NO: 11 - DNA, 16 bp, Artificial Sequence
  PDGFR-B forward primer
agcgctggcgaaatcg

SEQ ID NO: 12 - DNA, 19 bp, Artificial Sequence
  PDGFR-B reverse primer
ttcacgcgaaccagtgtca

SEQ ID NO: 13 - DNA, 21 bp, Artificial Sequence
  PDGFR-B probe
ctgtccacgcgcaacgtgtcg

SEQ ID NO: 14 - DNA, 23 bp, Artificial Sequence
  C-KIT forward primer
cagatttcagagagcaccaatca

SEQ ID NO: 15 - DNA, 21 bp, Artificial Sequence
  C-KIT reverse primer
aatggtctaccacgggcttct

SEQ ID NO: 16 - DNA, 30 bp, Artificial Sequence
  C-KIT probe
ttactccaacttagcaaactgcagccccaa

SEQ ID NO: 17 - DNA, 23 bp, Artificial Sequence
  C-RAF forward primer
gaggtcgacatccacacctaatg

SEQ ID NO: 18 - DNA, 22 bp, Artificial Sequence
  C-RAF reverse primer
tcgaattgcatcctcaatcatc

SEQ ID NO: 19 - DNA, 23 bp, Artificial Sequence
  C-RAF probe
ccacatggtcagcaccaccctgc

SEQ ID NO: 20 - DNA, 20 bp, Artificial Sequence
  EGFR forwrad primer
gaaggagctgcccatgagaa

SEQ ID NO: 21 - DNA, 21 bp, Artificial Sequence
  EGFR reverse primer
gactatgtcccgccactggat

SEQ ID NO: 22 - DNA, 21 bp, Artificial Sequence
  EGFR probe
aaatcctgcatggcgccgtgc

SEQ ID NO: 23 - DNA, 21 bp, Artificial Sequence
  mTOR forward primer
aggccgcattgtctctatcaa

SEQ ID NO: 24 - DNA, 24 bp, Artificial Sequence
  mTOR reverse primer
gcagtaaatgcaggtagtcatcca

SEQ ID NO: 25 - DNA, 22 bp, Artificial Sequence
  mTOR probe
tgcaatccagctgtttggcgcc

SEQ ID NO: 26 - DNA, 20 bp, Artificial Sequence
  FGFR1 forward primer
cacgggacattcaccacatc

SEQ ID NO: 27 - DNA, 19 bp, Artificial Sequence
  FGFR1 reverse primer
gggtgccatccacttcaca

SEQ ID NO: 28 - DNA, 30 bp, Artificial Sequence
  FGFR1 probe
actataaaaagacaaccaacggccgactgc

SEQ ID NO: 29 - DNA, 19 bp, Artificial Sequence
  B2M forward primer
cattcgggccgagatgtct

SEQ ID NO: 30 - DNA, 24 bp, Artificial Sequence
  B2M reverse primer
ctccaggccagaaagagagagtag

SEQ ID NO: 31 - DNA, 22 bp, Artificial Sequence
  B2M probe
ccgtggccttagctgtgctcgc

SEQ ID NO: 32 - DNA, 21 bp, Artificial Sequence
  GAPDH forward primer
cacatggcctccaaggagtaa

SEQ ID NO: 33 - DNA, 24 bp, Artificial Sequence
  GAPDH reverse primer
tgagggtctctctcttcctcttgt

SEQ ID NO: 34 - DNA, 22 bp, Artificial Sequence
  GAPDH probe
ctggaccaccagccccagcaag

SEQ ID NO: 35 - DNA, 20 bp, Artificial Sequence
  HMBS forward primer
ccagggatttgcctcacctt

SEQ ID NO: 36 - DNA, 21 bp, Artificial Sequence
  HMBS reverse primer
aaagagatgaagcccccacat

SEQ ID NO: 37 - DNA, 26 bp, Artificial Sequence
  HMBS probe
ccttgatgactgccttgcctcctcag

SEQ ID NO: 38 - DNA, 23 bp, Artificial Sequence
  HPRT1 forward primer
gctcgagatgtgatgaaggagat

SEQ ID NO: 39 - DNA, 21 bp, Artificial Sequence
  HPRT1 reverse primer
ccagcaggtcagcaaagaatt

SEQ ID NO: 40 - DNA, 28 bp, Artificial Sequence
  HPRT1 probe
ccatcacattgtagccctctgtgtgctc

SEQ ID NO: 41 - DNA, 20 bp, Artificial Sequence
  SDHA forward primer
cacctagtggctgggagctt

SEQ ID NO: 42 - DNA, 24 bp, Artificial Sequence
  SDHA reverse primer
gcccagttttatcatctcacaaga

SEQ ID NO: 43 - DNA, 27 bp, Artificial Sequence
  SDHA probe
tggcacttacctttgtcccttgcttca

(19) United States
(12) Patent Application Publication
(10) Pub. No.: US 2018/0030541 A1
(43) Pub. Date: Feb. 1, 2018

(54) METHOD FOR PREDICTING SENSITIVITY TO VASCULAR ENDOTHELIAL GROWTH FACTOR RECEPTOR INHIBITOR

(71) Applicants: CBSBIOSCIENCE INC., Daejeon (KR); GIL MEDICAL CENTER, Incheon (KR)

(72) Inventors: Jin Young Park, Seoul (KR); Young Ho Moon, Daejeon (KR); Jung-Hee Kwon, Daejeon (KR); Yun Soo Kim, Seoul (KR); Ju Hyun Kim, Incheon (KR); Duck Joo Choi, Seoul (KR)

(73) Assignees: CBSBIOSCIENCE INC., Daejeon (KR); GIL MEDICAL CENTER, Incheon (KR)

(21) Appl. No.: 15/521,566

(22) PCT Filed: Oct. 22, 2015
(86) PCT No.: PCT/KR2015/011238

§ 371 Date: Apr. 24, 2017

(30) Foreign Application Priority Data
Oct. 24, 2014 (KR) 10-2014-0144762

(51) Int. Cl. C12Q 1/68; A61K 31/44

(52) U.S. Cl. CPC C12Q 1/6886; A61K 31/44; C12Q 2600/136; C12Q 2600/158

(57) ABSTRACT

The present invention relates to a method for predicting sensitivity to treatment with vascular endothelial growth factor receptor (VEGFR) inhibitors, by using a biomarker. An effective means and anticancer therapy for cancer treatment of liver cancer are selected by predicting the effect of treatment with VEGFR inhibitors on liver cancer patients through the present invention, thereby increasing the treatment effect and minimizing the side effects of liver cancer treatment.

### TECHNICAL FIELD

The present invention relates to a method of predicting the sensitivity of a liver cancer patient to treatment with a vascular endothelial growth factor receptor inhibitor to determine whether or not the patient can receive a therapeutic effect from the treatment.

### BACKGROUND ART

Cancer is one of the most fatal diseases that pose a threat to human health. In the USA alone, cancer affects nearly 1.3 million new patients each year, and is the second leading cause of death after heart disease, accounting for approximately 1 in 4 deaths. Although there have been significant advances in the medical treatment of certain cancers, the overall 5-year survival rate for all cancers has improved only by about 10% in the past 20 years. Furthermore, cancers, or malignant tumors, metastasize and grow rapidly in an uncontrolled manner, making timely detection and treatment extremely difficult.

Depending on the cancer type, patients typically have several treatment options available to them including chemotherapy, radiation and antibody-based drugs, but the therapeutic effects of these treatment options differ depending on the patient and the nature of cancer.

Particularly, liver cancer is a carcinoma having high resistance to anticancer therapy, and many kinds of molecular targeted drugs have been studied for use as chemotherapeutic agents, but only some of these drugs have been approved by the FDA or the like and marketed. In addition, even approved and commercially available anticancer drugs against liver cancer show very low therapeutic effects depending on the nature of cancer in cancer patients and the lesion of cancer. For example, Nexavar (Sorafenib), a new drug approved for the purpose of treating hepatocellular carcinoma, shows a low treatment rate of about 2-3%, and the side effects thereof are also very high. Namely, current guidelines for treatment of hepatocellular carcinoma are not based on the molecular characteristics of tumors, and thus there is a limitation in that the therapeutic effects of the guidelines cannot be guaranteed. Furthermore, although some genes known as biomarkers are present, a threshold value and diagnostic performance for predicting resistance or sensitivity to these genes have not been properly evaluated, and thus the clinical usefulness of these genes needs to be demonstrated through additional studies.

Therefore, there is a need for research and development on a specific biomarker making it possible to determine whether an individual patient will respond to any treatment, and on a threshold value and diagnostic performance for predicting resistance or sensitivity by use of the biomarker. In addition, there is a need to develop a method that makes it possible to select anticancer therapy and that can achieve anticancer treatment by effectively introducing an appropriate anticancer drug into a patient.

### DISCLOSURE

### Technical Problem

The present invention is intended to provide a method for predicting sensitivity to a vascular endothelial growth factor receptor (VEGFR) inhibitor, the method comprising the steps of: measuring the expression levels of mTOR in samples isolated from liver cancer patients; and comparing the expression levels of mTOR with a reference level of mTOR expression, and selecting samples having an mTOR expression level equal to or higher than the reference level.

The present invention is also intended to provide a composition for predicting sensitivity to a vascular endothelial growth factor receptor (VEGFR) inhibitor, the composition containing an agent for measuring the mRNA expression level of mTOR.

The present invention is also intended to provide a kit for predicting sensitivity to a vascular endothelial growth factor receptor (VEGFR) inhibitor, the kit comprising the composition.

The present invention is also intended to provide a method of treating liver cancer using a vascular endothelial growth factor receptor (VEGFR) inhibitor, the method comprising the steps of: (a) measuring the expression levels of mTOR in samples isolated from liver cancer patients; (b) comparing the expression levels of mTOR with a reference level of mTOR expression; (b) selecting samples having an mTOR expression level equal to or higher than the reference level; and (c) administering a therapeutically effective amount of the vascular endothelial growth factor receptor (VEGFR) inhibitor to the liver cancer patients showing the mTOR expression level equal to or higher than the reference level.

### Technical Solution

The present inventors have made extensive efforts to select a patient having high sensitivity to a vascular endothelial growth factor receptor (VEGFR) inhibitor to thereby maximize the effect of anticancer treatment, and as a result, have found that, when the expression level of mTOR, VEGFR2, PDGFRβ, FGFR1, cKIT, EGFR or cRAF is measured, sensitivity to the vascular endothelial growth factor receptor (VEGFR) inhibitor can be predicted based on the expression level and treatment benefit score, thereby completing the present invention.

The present invention provides a method for predicting sensitivity to a vascular endothelial growth factor receptor (VEGFR) inhibitor, the method comprising the steps of: measuring the expression levels of mTOR in samples isolated from liver cancer patients; and comparing the expression levels of mTOR with a reference level of mTOR expression, and selecting samples having an mTOR expression level equal to or higher than the reference level.

In the present invention, the vascular endothelial growth factor receptor (VEGFR) inhibitor is a therapeutic agent that exhibits an anticancer effect by inhibiting vascular endothelial growth factor receptor (VEGFR), and it may be, for example, an anticancer agent, such as brivanib, sunitinib, linifanib, regorafenib, sorafenib or the like. According to one embodiment of the present invention, the vascular endothelial growth factor receptor (VEGFR) inhibitor is sorafenib.

As used herein, the term “sensitivity” refers to the property capable of benefiting or providing a therapeutic effect from treatment with a vascular endothelial growth factor receptor (VEGFR) inhibitor that is a targeted anticancer drug. For example, the term means that the tumor size is reduced by 5%, 10,% 15%, 20%, 25%, 30% or more after anticancer treatment. Preferably, the term means that the tumor size is reduced by 30% or more (complete response and partial response rates according to RECIST (Response Evaluation Criteria in Solid Tumors), criteria for evaluating the response of solid tumors to anticancer agents).

Namely, according to the present invention, sensitivity to a vascular endothelial growth factor receptor (VEGFR) inhibitor is predicted, thereby predicting the possibility of treatment with the vascular endothelial growth factor receptor (VEGFR) inhibitor and the prognosis of the treatment and selecting an effective means and anticancer therapy for anticancer treatment to thereby increase therapeutic effects and minimize the side effects of cancer treatment.

In particular, the present invention have advantages over conventional inventions in that it can exhibit the effect of sorafenib treatment, showing high accuracy by measuring the expression level of a biomarker of mTOR alone, and in that an increase in the mRNA level of mTOR shows high sensitivity to sorafenib treatment. Furthermore, the present invention shows the results of predicting a high level of sensitivity by examining sensitivity directly in patient samples, not in cell lines.

The method for predicting sensitivity to the vascular endothelial growth factor receptor (VEGFR) inhibitor according to the present invention comprises measuring a step of measuring the expression levels of mTOR in samples isolated from liver cancer patients.

As used herein, the tem “liver cancer patients” refers to patients having primary liver cancer that is a malignant tumor occurring in the liver. Preferably, the term refers to patients having hepatocellular carcinoma among hepatocellular carcinoma and intrahepatic bile duct cancer, which are primary liver cancers. In addition, the samples include whole blood, plasma, serum, tissue or the like isolated from liver cancer patients. According to one embodiment of the present invention, liver tumor tissues are used. These samples may be suitably processed according to methods known in the art in order to measure expression levels.

In the present invention, “measuring the expression levels” includes measuring mRNA expression levels. In the present invention, “measuring mRNA expression levels” is a process of examining the presence and expression level of mRNA of a gene in samples in order to predict sensitivity to the vascular endothelial growth factor receptor (VEGFR) inhibitor, and may be performed by measuring the mRNA levels. Assay methods for this measurement include, but are not limited to, RT-PCR, competitive RT-PCR, real-time RT-PCR, RNase protection assay (RPA), Northern blotting, DNA chip assay, etc.

According to one embodiment of the present invention, the mRNA expression levels are measured by real-time RT-PCR. Furthermore, the mRNA expression level can be quantified as 2^{-ΔCt} by measuring the CT value of (the number of cycles required to reach the threshold value) of each gene and calculating the ΔCT value (CT of each marker minus the average CT value of a reference gene). In the present invention, the mTOR expression levels are measured in order to predict sensitivity to the vascular endothelial growth factor receptor (VEGFR) inhibitor. mTOR (mammalian target of rapamycin) is a serine/threonine kinase gene that is targeted by rapamycin in mammals, and has a nucleotide sequence of SEQ ID NO: 1.

As used herein, the term “reference level” refers to the same level as the expression level of genes capable of showing high diagnostic predictability detected and identified from reference samples by the method described herein. The reference level can be relative to a number or value derived from population studies, including subjects having the same cancer, subjects having the same or similar age range, subjects in the same or similar ethnic group, subjects having family histories of cancer, or relative to the starting sample of a subject undergoing treatment for cancer. Such reference levels can be derived from statistical analyses and/or risk prediction data of populations obtained from mathematical algorithms and computed indices. Reference indices can also be constructed and used using algorithms and other methods of statistical and structural classification.

In certain embodiments, the term “reference level” herein refers to a predetermined value. For example, a person skilled in the art will appreciate that the reference level is predetermined and set to meet routine requirements in terms of specificity, sensitivity and/or accuracy. For example, sensitivity or specificity, respectively, has to be set to certain limits, e.g. 80%, 90% or 95%, respectively. These requirements may also be defined in terms of positive or negative predictive values. The reference level may be predetermined in reference samples from healthy individuals or predetermined from the disease entity to which the patient belongs.

In the present invention, a statistical analysis method such as mean value calculation or ROC curve analysis may be used to determine the reference level of expression.

ROC curve analysis according to one embodiment of the present invention is performed in terms of sensitivity, specificity and accuracy using a curve that shows the performance of diagnosis. In the present invention, sensitivity is the ratio of true positives to the sum of persons having sensitivity to a vascular endothelial growth factor receptor (VEGFR) inhibitor; specificity is the ratio of true negatives to the sum of persons having no sensitivity to a vascular endothelial growth factor receptor (VEGFR) inhibitor; and accuracy is the ratio of hits to all cases. When both specificity and sensitivity are high, the accuracy of test results increases. Thus, the x-axis in the ROC curve is 1-specificity, and the y-axis is sensitivity, and the AUC (area under curve) indicating accuracy means the area under the curve.

According to one embodiment of the present invention, “reference level” is a threshold value that shows a high predictive effect on the prediction of sensitivity to a vascular endothelial growth factor receptor (VEGFR) inhibitor. For the prediction of sensitivity, the expression level of mTOR, which corresponds to an AUC value higher than the average level (about 0.6), preferably the highest AUC value, in the ROC curve analysis of mTOR, is selected as the best threshold value (criterion), and a sample having a mTOR expression level equal to or higher than the reference level is predicted to have high sensitivity to a vascular endothelial growth factor receptor (VEGFR) inhibitor. For example, the reference level for the mRNA expression level according to one embodiment of the present invention may be 0.0007 (criterion), preferably 0.0004.

As used herein, the term “more than” or “higher than” means a level higher than the reference level or means an overall increase of 1%, 2%, 5%, 10%, 20%, 25%, 30%, 40%, 50%, 60%, 70%, 80%, 85%, 90%, 95%, 96%, 97%, 98%, 99% or more in the expression level detected by the method described herein, compared to the expression level in the reference sample. As used herein, the term “less than” or “lower than” means a level lower than the reference level or means an overall decrease of 1%, 2%, 5%, 10%, 20%, 25%, 30%, 40%, 50%, 60%, 70%, 80%, 85%, 90%, 95%, 96%, 97%, 98%, 99% or more in the expression level detected by the method described herein, compared to the expression level in the reference sample.

The method according to the present invention may further comprises the steps of: measuring the expression levels of one or more biomarkers, selected from among VEGFR2 and PDGFRβ, together with measurement of the expression level of mTOR; and comparing the expression level of each of the biomarkers with reference levels for the biomarkers together with comparing the mTOR expression level with the reference level, and selecting samples having expression levels equal to or higher than the reference levels of the biomarkers.

In the present invention, VEGFR2 (vascular endothelial growth factor receptor 2) is a tyrosine kinase-encoding gene acting as the receptor of vascular endothelial growth factor (VEGF) A, C, D and E, and has a nucleotide sequence of SEQ ID NO: 2.

In the present invention, PDGFRβ (platelet-derived growth factor receptor β) is a tyrosine kinase-encoding gene acting as the receptor of platelet-derived growth factor (PDGF), and has a nucleotide sequence of SEQ ID NO: 3.

Preferably, the biomarkers used together with mTOR in the method for predicting sensitivity are VEGFR2 and PDGFRβ.

In the method for predicting sensitivity, a treatment benefit score (TBS) can be calculated from the expression level of mTOR, and sensitivity to the vascular endothelial growth factor receptor (VEGFR) inhibitor can be predicted therefrom. Preferably, sensitivity to the vascular endothelial growth factor receptor (VEGFR) inhibitor can be predicted from a treatment benefit score (TBS) obtained by measuring the expression levels of mTOR and one or more selected from among VEGFR2 and PDGFRβ and calculating the sum of the expression levels. In the present invention, “treatment benefit score” is a value obtained from information indicating that a patient can receive a therapeutic effect from the vascular endothelial growth factor receptor (VEGFR) inhibitor. If a patient has a treatment benefit score equal to or higher than a certain score, determined based on the reference level, the patient has sensitivity to sorafenib. According to one embodiment of the present invention, the treatment benefit score can be calculated by multiplying the sum of mRNA expression levels, quantified as 2^{-ΔCt}, by 100. Herein, to determine the reference level, the AUC value in ROC curve analysis of the treatment benefit score, which is equal to higher than an average level (about 0.6), preferably the highest AUC value, is used as the best threshold value (criterion).

The present invention also provides a composition for predicting sensitivity to a vascular endothelial growth factor receptor (VEGFR) inhibitor, the composition containing an agent for measuring the mRNA expression level of mTOR.

The composition for predicting sensitivity to the vascular endothelial growth factor receptor (VEGFR) inhibitor makes it possible to predict sensitivity to the vascular endothelial growth factor receptor (VEGFR) inhibitor by measuring the mRNA expression level of mTOR.

Preferably, the composition for predicting sensitivity to the vascular endothelial growth factor receptor (VEGFR) inhibitor may further contain an agent for measuring the mRNA expression level of at least one biomarker selected from among VEGFR2 and PDGFRβ. More preferably, the composition for predicting sensitivity to the vascular endothelial growth factor receptor (VEGFR) inhibitor further contains agents for measuring the mRNA expression levels of VEGFR2 and PDGFRβ.

Preferably, the composition for predicting sensitivity to the vascular endothelial growth factor receptor (VEGFR) inhibitor may contain any one or more of a primer pair, a probe and an anti-sense nucleotide, which are agents for measuring mRNA expression levels and bind specifically to the genes. The sequence of the above-described primer pair, probe or antisense nucleotide can be easily designed by those skilled in the art from the nucleotide sequences provided in the present invention. According to one embodiment of the present invention, the agent for measuring the mRNA expression level comprises a primer pair and a probe, and may have the primer pair and probe sequences of mTOR, VEGFR2 and/or PDGFRβ shown in Table 1 below.

The present invention also provides a kit for predicting sensitivity to a vascular endothelial growth factor receptor (VEGFR) inhibitor, the kit containing a composition for predicting sensitivity to the vascular endothelial growth factor receptor (VEGFR) inhibitor.

The kit for predicting sensitivity to a vascular endothelial growth factor receptor (VEGFR) inhibitor may further contain an agent for measuring the mRNA expression level of at least one biomarker selected from among VEGFR2 and PDGFRβ. More preferably, the for predicting sensitivity to the vascular endothelial growth factor receptor (VEGFR) inhibitor further contains agents for measuring the mRNA expression levels of VEGFR2 and PDGFRβ.

The kit makes it possible to predict sensitivity to the vascular endothelial growth factor receptor (VEGFR) inhibitor by measuring the expression level of any one or more of polynucleotides encoding the proteins. The kit for predicting sensitivity to the vascular endothelial growth factor receptor (VEGFR) inhibitor according to the present invention may contain not only a primer pair or a probe for measuring expression levels, but also one or more other constituent compositions or devices suitable for analysis of the polynucleotides. Preferably, the diagnostic kit for quantitative detection of the above-described polynucleotides or genes according to the present invention may contain one or more oligonucleotides that bind specifically to the polynucleotides encoding the four proteins. Specifically, the diagnostic kit may contain primers corresponding to the oligonucleotides, reverse transcriptase, Taq polymerase, primers for PCR, and dNTP. To measure polynucleotide expression levels, a kit employing an analytical method described with respect to “the measurement of mRNA expression levels” may be used. For example, the kit may be selected from among RT-PCR kits, competitive RT-PCR kits, real-time RT-PCR kits, real-time RT-PCR kits, and DNA chip kits.

The present invention also provides a method of treating liver cancer using a vascular endothelial growth factor receptor (VEGFR) inhibitor, the method comprising the steps of: (a) measuring the expression levels of mTOR in samples isolated from liver cancer patients; (b) comparing the expression levels of mTOR with a reference level of mTOR expression, and selecting samples having an mTOR expression level equal to or higher than the reference level; and (c) administering a therapeutically effective amount of the vascular endothelial growth factor receptor (VEGFR) inhibitor to the liver cancer patients showing the mTOR expression level equal to or higher than the reference level.

In addition, the method for treating liver cancer may further comprise: measuring the expression levels of mTOR and at least one selected from VEGFR2 and PDGFRβ; and calculating the sum of the expression levels, and selecting samples in which the sum of the expression levels is equal to or higher than a reference level for the sum of the expression levels. More preferably, the biomarkers, the levels of which are measured together with the level of mTOR and compared with the reference level, are VEGFR2 and PDGFRβ.

Based on the sum of the expression levels (preferably mRNA expression levels of mTOR and the biomarker, samples having a treatment benefit score equal to or higher than the reference level are selected. After selection of the samples, a therapeutically effective amount of the vascular endothelial growth factor receptor (VEGFR) inhibitor may be administered to a liver cancer patient showing the treatment benefit score equal to or higher than the reference level.

The vascular endothelial growth factor receptor (VEGFR) inhibitor is any one or more selected from among brivanib, sunitinib, linifanib, regorafenib, and sorafenib. Furthermore, the method may further comprise a step of administering a therapeutically effective amount of a therapeutic agent selected from the group consisting of cytotoxic agents, chemotherapeutic agents, growth inhibitors, antiangiogenic agents, and combinations thereof.

The present invention also provides the use of an agent for measuring the expression level of mTOR, for prediction of sensitivity to a vascular endothelial growth factor receptor (VEGFR) inhibitor. The use according to the present invention may further comprise, in addition to the use of the agent for measuring the expression level of mTOR, the use of at least one biomarker selected from VEGFR2 and PDGFRβ. Preferably, the agent comprises agents for measuring the expression levels of VEGFR2 and PDGFRβ.

The present invention also provides a method for predicting a vascular endothelial growth factor receptor (VEGFR) inhibitor, the method comprising the steps of: measuring the expression levels of FGFR1, cKIT, EGFR (epidermal growth factor receptor) or cRAF in samples isolated from liver cancer patients; and comparing the expression levels of FGFR1, cKIT, EGFR or cRAF with a reference level of expression of each of the these biomarkers, and selecting samples having an expression level higher than the reference level.

The present invention also provides a composition for predicting a vascular endothelial growth factor receptor (VEGFR) inhibitor, the composition containing an agent for measuring the mRNA expression level of FGFR1, cKIT, EGFR or cRAF.

The present invention also provides a kit for predicting a vascular endothelial growth factor receptor (VEGFR) inhibitor, the kit containing the composition for predicting the vascular endothelial growth factor receptor (VEGFR) inhibitor.

The present invention also provides a method for treating liver cancer using a vascular endothelial growth factor receptor (VEGFR) inhibitor, the method comprising the steps of: (a) measuring the expression levels of FGFR1, cKIT, EGFR or cRAF in samples isolated from liver cancer patients; (b) comparing the expression levels of FGFR1, cKIT, EGFR or cRAF with a reference level of expression of each of the these biomarkers, and selecting samples having an expression level higher than the reference level; and (c) administering a therapeutically effective amount of the vascular endothelial growth factor receptor (VEGFR) inhibitor to the selected liver cancer patients.

The present invention also provides the use of an agent for measuring the expression level of FGFR1, cKIT, EGFR or cRAF, for predicting sensitivity to a vascular endothelial growth factor receptor (VEGFR) inhibitor.

In the present invention FGFR1 (fibroblast growth factor receptor 1) is a tyrosine kinase-encoding gene acting as the receptor of fibroblast growth factor (FGF), and has a nucleotide sequence of SEQ ID NO: 4.

In the present invention, cKIT is a tyrosine kinase-encoding gene acting as the receptor of stem cell factor (SCF), and has a nucleotide sequence of SEQ ID NO: 5.

In the present invention, EGFR (epidermal growth factor receptor) is a tyrosine kinase-encoding gene acting as the receptor of epidermal growth factor (EGF), and has a nucleotide sequence of SEQ ID NO: 6.

In the present invention, cRAF is a kinase-encoding gene belonging to the family of RAF serine/threonine kinases well-known as oncogenes, and has a nucleotide sequence of SEQ ID NO: 7.

The matters mentioned in the prediction method, composition, kit, treatment method and use of the present invention are applied in the same manner unless they are not contradictory to each other.

### Advantageous Effects

According to the present invention, effects on the treatment of liver cancer patients with a vascular endothelial growth factor receptor (VEGFR) inhibitor are predicted. Based on the prediction, an effective means and anticancer therapy for treatment of liver cancer are selected, thereby increasing treatment effects and minimizing the side effects of liver cancer treatment.

### DESCRIPTION OF DRAWINGS

FIG. 1 shows the results of analyzing the expression level distributions of mTOR, VEGFR2, PDGFRβ, FGFR1, cKIT, EGFR or cRAF in responders and non-responders to sorafenib.

FIG. 2 shows the results of analyzing the treatment benefit score distribution of responders to sorafenib and non-responders to sorafenib by use of mTOR, a combination of mTOR and VEGFR2, a combination of mTOR and PDGFRβ, or a combination of mTOR, VEGFR2 and PDGFRβ.

FIG. 3 shows the results of receiver operating characteristic (ROC) analysis performed to confirm the ability of treatment benefit scores to predict and classify responders to sorafenib and non-responders to sorafenib.

### MODE FOR INVENTION

The advantages and features of the present invention, and the way of attaining them, will become apparent with reference to the examples described below. However, the present invention is not limited to the examples disclosed below and can be embodied in a variety of different forms; rather, these examples are provided so that this disclosure will be thorough and complete, and will fully convey the scope of the present invention to those skilled in the art. The scope of the present invention will be defined by the appended claims.

### Example 1: RNA Extraction and cDNA Synthesis

From 31 liver cancer patients who were diagnosed as having liver cancer, received hepatectomy or liver transplantation, showed recurrence of liver cancer, and received treatment with Nexavar (sorafenib) in Ajou University Medical Center, Korea University Anam Hospital and Keimyung University Dongsan Medical Center, liver cancer tissues were obtained under informed consent. In the following manner, RNA was extracted from each of the tissues and cDNA was synthesized therefrom.

Total RNA was extracted from liver cancer tissue and the surrounding normal tissue using an RNeasy Mini kit (Qiagen, Germany) according to the manufacturer's instructions. The extracted total RNA was quantified using Bioanalyzer 2100 (Agilent Technologies, USA). In the extraction step, the RNA extract was treated with DNase I to remove genomic DNA. Each sample comprising 4 μg of total RNA was incubated with 2 μl of 1 μM oligo d(T)18 primer (Genotech, Korea) at 70° C. for 7 minutes and cooled on ice for 5 minutes. A total of 11 μl of an enzyme mixture [2 μl of 0.1 M DTT (Duchefa, the Netherlands), 2 μl of 10× reverse transcription buffer, 5 μl of 2 mM dNTP, 1 μl of 200 U/μl MMLV reverse transcriptase and 1 μl of 40 U/μl RNase inhibitor (Enzynomics, Korea)] was separately prepared. The enzyme mixture was added to the mixture containing the RNA, and was incubated at 42° C. for 90 minutes, and then incubated at 80° C. for 10 minutes to inactivate the reverse transcriptase. Diethyl pyrocarbonate (DSPC)-treated water was added to the mixture to a final volume of 400 μl.

### Example 2: Quantitative Real-Time PCR

In order to measure the mRNA expression level of mTOR, VEGFR2, PDGFRβ, FGFR1, cKIT, EGFR or cRAF, for each of the cDNA samples obtained in Example 1, the gene markers shown in Table below were amplified by real-time PCR using PRISM 7900HT (Applied Biosystems, USA) according to the manufacturer's instructions.

Real-time PCR analysis was performed using a total of 10 μl of a volume consisting of 5 μl of 2× TaqMan gene expression master mix (Applied Biosystems, USA), 1 μl of each of 5 μM forward and reverse primers, 1 μl of 1 μM probe (Genotech, Korea) and 2 μl of cDNA (the same amount of water as control). The PCR amplification was performed by denaturation at 95° C. for 10 minutes, followed by cycle reactions consisting of denaturation at 95° C. for 15 sec and synthesis at 60° C. for 1 minute. The primer and probe sequences were designed using Primer Express 3.0 (Applied Biosystems, USA), and all the probe sequences were labeled with FAM at the 5′ end and TAMRA at the 3′ end. For each of the markers, the primer and probe sequences shown in Table 1 below were used.

TABLE 1
Information of primer and probe sequences for
marker genes

| Genes | | Sequences |
|---|---|---|
| VEGFR2 | F | CACCACTCAAACGCTGACATGTA (SEQ ID |
| | | NO: 8) |
| | R | CCAACTGCCAATACCAGTGGAT (SEQ ID NO: 9) |
| | P | TATGCCATTCCTCCCCCGCATCA (SEQ ID |
| | | NO: 10) |
| PDGFR-β | F | AGCGCTGGCGAAATCG (SEQ ID NO: 11) |
| | R | TTCACGCGAACCAGTGTCA (SEQ ID NO: 12) |
| | P | CTGTCCACGCGCAACGTGTCG (SEQ ID NO: 13) |
| C-KIT | F | CAGATTTCAGAGAGCACCAATCA (SEQ ID |
| | | NO: 14) |
| | R | AATGGTCTACCACGGGCTTCT (SEQ ID NO: 15) |
| | P | TTACTCCAACTTAGCAAACTGCAGCCCCAA |
| | | (SEQ ID NO: 16) |
| C-RAF | F | GAGGTCGACATCCACACCTAATG (SEQ ID |
| | | NO: 17) |
| | R | TCGAATTGCATCCTCAATCATC (SEQ ID |
| | | NO: 18) |
| | P | CCACATGGTCAGCACCACCCTGC (SEQ ID |
| | | NO: 19) |
| EGFR | F | GAAGGAGCTGCCCATGAGAA (SEQ ID NO: 20) |
| | R | GACTATGTCCCGCCACTGGAT (SEQ ID NO: 21) |
| | P | AAATCCTGCATGGCGCCGTGC (SEQ ID NO: 22) |
| mTOR | F | AGGCCGCATTGTCTCTATCAA (SEQ ID NO: 23) |
| | R | GCAGTAAATGCAGGTAGTCATCCA (SEQ ID |
| | | NO: 24) |
| | P | TGCAATCCAGCTGTTTGGCGCC (SEQ ID |
| | | NO: 25) |
| FGFR1 | F | CACGGGACATTCACCACATC (SEQ ID NO: 26) |
| | R | GGGTGCCATCCACTTCACA (SEQ ID NO: 27) |
| | P | ACTATAAAAAGACAACCAACGGCCGACTGC |
| | | (SEQ ID NO: 28) |

The expression of each marker gene was repeatedly measured three times, and normalized to the average expression of five reference genes (B2M, GAPDH, reference genes, the primer and probe sequences shown in Table 2 below were used.

TABLE 2
Information of primer and probe sequences for
reference genes

| Genes | Sequences | |
|---|---|---|
| B2M | F | CATTCGGGCCGAGATGTCT (SEQ ID NO: 29) |
| | R | CTCCAGGCCAGAAAGAGAGAGTAG (SEQ ID NO: 30) |
| | P | CCGTGGCCTTAGCTGTGCTCGC (SEQ ID NO: 31) |
| GAPDH | F | CACATGGCCTCCAAGGAGTAA (SEQ ID NO: 32) |
| | R | TGAGGGTCTCTCTCTTCCTCTTGT (SEQ ID NO: 33) |
| | P | CTGGACCACCAGCCCCAGCAAG (SEQ ID NO: 34) |
| HMBS | F | CCAGGGATTTGCCTCACCTT (SEQ ID NO: 35) |
| | R | AAAGAGATGAAGCCCCCACAT (SEQ ID NO: 36) |
| | P | CCTTGATGACTGCCTTGCCTCCTCAG (SEQ ID |
| | | NO: 37) |
| HPRT1 | F | GCTCGAGATGTGATGAAGGAGAT (SEQ ID NO: 38) |
| | R | CCAGCAGGTCAGCAAAGAATT (SEQ ID NO: 39) |
| | P | CCATCACATTGTAGCCCTCTGTGTGCTC (SEQ ID |
| | | NO: 40) |
| SDHA | F | CACCTAGTGGCTGGGAGCTT (SEQ ID NO: 41) |
| | R | GCCCAGTTTTATCATCTCACAAGA (SEQ ID NO: 42) |
| | P | TGGCACTTACCTTTGTCCCTTGCTTCA (SEQ ID |
| | | NO: 43) |

The CT value (the number of cycles required to reach a threshold value) of each marker was measured, and the ΔCT value (the CT value of each marker minus the mean CT value of reference genes) was calculated, thereby the mRNA expression level of each marker was expressed as 2^{-ΔCt}.

### Example 3: Statistical Analysis

Using the expression levels (2^{-ΔCt}) of the markers obtained in Example 2, a total of four statistical analyses (i.e., individual gene expression distribution analysis, ROC curve analysis, treatment benefit score analysis, and ROC curve analysis of treatment benefit scores) were performed in the following manner.
(1) Analysis of Expression Distributions of Individual Genes

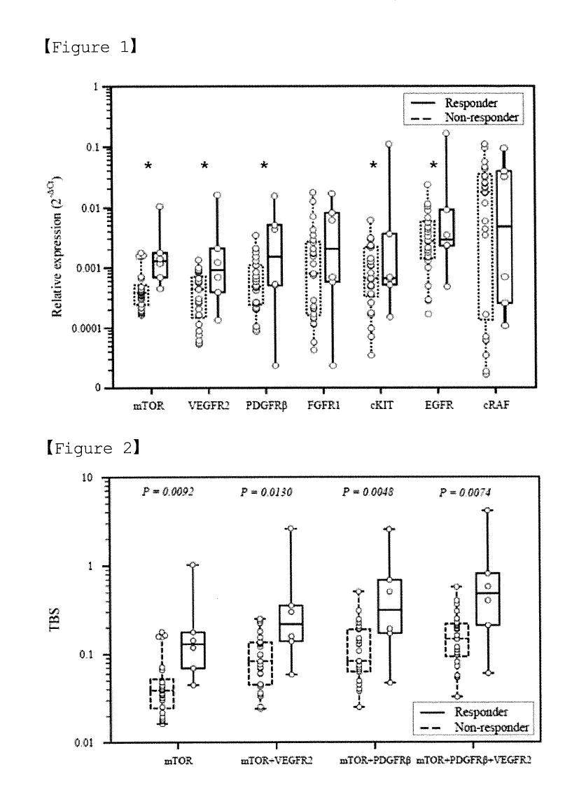

In order to examine the correlation between the therapeutic effect of sorafenib and the expression levels (2^{-ΔCT}) of the individual markers, the expression levels of the seven individual markers and the distribution thereof were analyzed for responders to sorafenib and non-responders to sorafenib, respectively, and the results of the analysis are shown in FIG. 1.

FIG. 1 shows the results of analyzing the mRNA expression levels of seven genes (i.e., mTOR, VEGFR2, PDGFRβ, FGFR1, cKIT, EGFR and cRAF) for responders and non-responders.

When expression of each of these markers showed that the expression level of mTOR was up-regulated in the responders (mean value of 2^{-ΔCT}: 0.0026) compared to in the non-responders (mean value of 2^{-ΔCT}: 0.0003), with statistical significance (P=0.0092). Furthermore, it was shown that the expression level of VEGFR2 was up-regulated in the responders (mean value of 2^{-ΔCT}: 0.0034) compared to in the non-responders (mean value of 2^{-ΔCT}: 0.0001), with statistical significance (P=0.0181). It was also shown that the expression level of PDGFRβ was up-regulated in the responders (mean value of 2^{-ΔCT}: 0.0043) compared to in the non-responders (mean value of 2^{-ΔCT}: 0.0001), with statistical significance (P=0.0043). Moreover, it was shown that the expression level of FGFR1 was up-regulated in the responders (mean value of 2^{-ΔCT}: 0.0053) compared to in the non-responders (mean value of 2^{-ΔCT}: 0.0002).

In addition, it was shown that the expression level of cKIT was up-regulated in the responders (mean value of 2^{-ΔCT}: 0.0192) compared to in the non-responders (mean value of 2^{-ΔCT}: 0.0005), with statistical significance (P=0.0408). Furthermore, it was shown that the expression level of EGFR was up-regulated in the responders (mean value of 2^{-ΔCT}: 0.0302) compared to in the non-responders (mean value of 2^{-ΔCT}: 0.0022), with statistical significance (P=0.0495). It was also shown that the expression level of cRAF was down-regulated in the responders (mean value of 2^{-ΔCT}: 0.0276) compared to in the non-responders (mean value of 2^{-ΔCT}: 0.0310), with statistical significance (P=0.8089).

(2) ROC Curve Analysis

In order to confirm whether the expression levels of individual markers in the 31 patients, calculated as described above, have the ability to predict and classify responders to sorafenib and non-responders to sorafenib, ROC (receiver operating characteristic) curve analysis was performed using various cut-off values.

Furthermore, based on the gene expression level corresponding to the average AUC value (about 0.6) or the highest AUC value in the ROC curve, the best threshold value (criterion) was obtained, and sensitivity, specificity, AUC value and p value, which correspond to each classifier and criterion, were obtained. The results of the analysis are shown in Table 3 below.

TABLE 3
Sensitivity, specificity and accuracy for prediction of
the response to sorafenib treatment of each gene

| | | Sensitivity | Specificity | | |
|---|---|---|---|---|---|
| Genes | Criterion | (%) | (%) | AUC | P value |
| mTOR | >0.0007 | 66.67 | 84.0 | 0.65 | 0.041 |
| | >0.0004 | 100.0 | 72.0 | 0.860 | <0.0001 |
| VEGFR2 | >0.0004 | 66.67 | 48.0 | 0.57 | 0.082 |
| | >0.001 | 50.0 | 96.0 | 0.740 | 0.0761 |
| PDGFRβ | >0.0005 | 83.33 | 48.0 | 0.63 | 0.261 |
| | >0.0035 | 50.0 | 100.0 | 0.683 | 0.2723 |
| FGFR1 | >0.0006 | 66.67 | 44.0 | 0.55 | 0.630 |
| | >0.0042 | 50.0 | 88.0 | 0.607 | 0.510 |
| cKIT | >0.0007 | 50.0 | 48.00 | 0.511 | 0.54 |
| | >0.0031 | 33.3 | 96.0 | 0.587 | 0.559 |
| EGFR | >0.0015 | 83.33 | 28.0 | 0.55 | 0.539 |
| | >0.0022 | 83.3 | 48.0 | 0.587 | 0.5346 |
| cRaf | >0.0003 | 66.67 | 28.0 | 0.514 | 0.719 |
| | >0.0001 | 100.0 | 24.0 | 0.533 | 0.810 |

Table 3 above shows the results of ROC curve analysis performed to predict a response to sorafenib based on the mRNA expression level of each marker gene. The results of the ROC curve analysis indicated that the highest AUC was 0.860 (p<0.0001) in mTOR. Particularly, it was shown that the AUC value was higher in mTOR, VEGRF2 and PDGFRβ than in other markers.

(3) Calculation of Treatment Benefit Score

In order to examine the correlation between the therapeutic effect of sorafenib and treatment benefit scores based on the expression levels of mTOR, VEGFR2 and/or PDGFRβ, the treatment benefit scores were calculated.

The treatment benefit score was calculated by selecting and combining the markers having high AUC values as described above, calculating the sum of the expression levels of the selected markers, and multiplying the sum by 100.

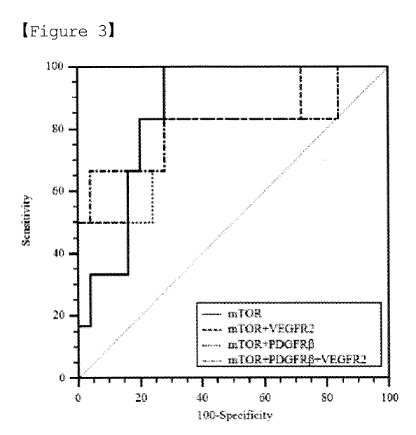

The results of calculating the treatment benefit scores based on a combination of mTOR, VEGRF2 and PDGFRβ, which showed high AUC values, are shown in FIG. 2 for responders and non-responders.

As shown in FIG. 2, the treatment benefit score in the case of mTOR was up-regulated in the responders (mean value of treatment benefit scores (TBS): 0.03) compared to in the non-responders (mean value of treatment benefit scores (TBS): 0.26), with statistical significance (P=0.0092). Furthermore, it was shown that the treatment benefit score obtained using a combination of mTOR and VEGRF2 was up-regulated in the responders (mean value of treatment benefit scores (TBS): 0.61) compared to in the non-responders (mean value of treatment benefit scores (TBS): 0.04), with statistical significance (P=0.0130). It was also shown that the treatment benefit score obtained using a combination of mTOR and PDGFRβ was up-regulated in the responders (mean value of treatment benefit scores (TBS): 0.69) compared to in the non-responders (mean value of treatment benefit scores (TBS): 0.05), with statistical significance (P=0.0048). In addition, it was shown that the treatment benefit score obtained using a combination of mTOR, VEGRF2 and PDGFRβ was up-regulated in the responders (mean value of treatment benefit scores (TBS): 1.04) compared to in the non-responders (mean value of treatment benefit scores (TBS): 0.05), with statistical significance (P=0.0074).

(4) ROC Curve Analysis of Treatment Benefit Scores

In order to evaluate the ability of the above-described treatment benefit scores to predict the therapeutic effect of sorafenib, ROC (receiver operating characteristic) analysis was performed using various cut-off values. Furthermore, based on the gene expression level corresponding to the average AUC value (about 0.6) or the highest AUC value in the ROC curve, the best threshold value (criterion) was obtained, and sensitivity, specificity, AUC value and p value, which correspond to each classifier and criterion, were obtained. The results of the analysis are shown in FIG. 3 and Table 4 below.

TABLE 4
Sensitivity, specificity and accuracy of prediction of sorafenib
treatment response, obtained based on treatment benefit scores

| | | Sensitivity | Specificity | | |
|---|---|---|---|---|---|
| Genes | Criterion | (%) | (%) | AUC | P value |
| mTOR | >0.07 | 66.67 | 84.0 | 0.65 | 0.041 |
| | >0.04 | 100.00 | 72.0 | 0.860 | <0.0001 |
| mTOR + | >0.18 | 50.0 | 88.0 | 0.61 | 0.028 |
| VEGFR2 | >0.14 | 83.33 | 80.0 | 0.820 | 0.0070 |
| mTOR + | >0.19 | 50.0 | 76.0 | 0.62 | 0.0312 |
| PDGFRβ | >0.15 | 83.33 | 72.0 | 0.773 | 0.0472 |
| mTOR + | >0.15 | 83.3 | 48.0 | 0.643 | 0.0208 |
| VEGFR2 + | >0.4 | 66.7 | 96.0 | 0.807 | 0.0274 |
| PDGFRβ | | | | | |

FIG. 3 and Table 4 above show the ROC curve based on the treatment benefit score obtained using mTOR alone or in combination with VEGRF2 and/or PDGFRβ. In the results of the ROC analysis based on the treatment benefit score obtained using mTOR alone or in combination with VEGRF2 and/or PDGFRβ, the AUC values were 0.860 when mTOR was used alone, 0.820 when mTOR was used in combination with VEGRF2, 0.773 when mTOR was used in combination with PDGFRβ, and 0.807 when mTOR was used in combination with VEGRF2 and PDGFRβ.

As can be seen from the foregoing, the markers and prediction method of the present invention show significant effects on prediction of the therapeutic effect of sorafenib. In particular, the therapeutic effect of sorafenib can be more accurately predicted from the treatment benefit scores obtained from mTOR and combinations of mTOR and additional markers.

1. A method for predicting sensitivity to a vascular endothelial growth factor receptor (VEGFR) inhibitor, the method comprising the steps of:
(a) measuring expression levels of mTOR in samples isolated from liver cancer patients; and
(b) comparing the expression levels of mTOR with a reference level of mTOR expression, and selecting samples having the expression level of mTOR equal to or higher than the reference level.

2. The method of claim 1, wherein the step (a) further comprises measuring expression levels of one or more biomarkers selected from among VEGFR2 and PDGFRβ; and
the step (b) further comprises comparing the expression levels of the one or more biomarkers selected from among VEGFR2 and PDGFRβ, with a reference level of expression of each of the biomarkers, and selecting samples having the biomarker expression level equal to or higher than the reference level.

3. The method of claim 1, wherein the vascular endothelial growth factor receptor (VEGFR) inhibitor is any one or more selected from among brivanib, sunitinib, linifanib, regorafenib, and sorafenib.

4. The method of claim 1, wherein the step (a) of measuring expression levels is measuring mRNA expression levels.

5. The method of claim 4, wherein the step (a) of measuring mRNA expression levels is measuring mRNA expression levels of mTOR and one or more biomarkers selected from among VEGFR2 and PDGFRβ.

6. The method of claim 1, wherein the reference level is 0.6 or more as an AUC value.

7. The method of claim 1, wherein the reference level is 0.0007 or more based on the mRNA expression level of mTOR.

8. The method of claim 1, wherein the mTOR has a nucleotide sequence of SEQ ID NO: 1.

9. A composition for predicting sensitivity to a vascular endothelial growth factor receptor (VEGFR) inhibitor, the composition containing an agent for measuring the mRNA expression level of mTOR.

10. The composition of claim 9, wherein the composition further contains an agent for measuring mRNA expression levels of one or more biomarkers selected from among VEGFR2 and PDGFRβ.

11. The composition of claim 9, wherein the vascular endothelial growth factor receptor (VEGFR) inhibitor is any one or more selected from among brivanib, sunitinib, linifanib, regorafenib, and sorafenib.

12. The composition of claim 9, wherein the agent for measuring the mRNA expression level is a primer pair, a probe or an antisense nucleotide, which binds specifically to the genes.

13. A kit for predicting sensitivity to a vascular endothelial growth factor receptor (VEGFR) inhibitor, the kit containing the composition of claim 9.

14. The kit of claim 13, wherein the kit is an RT-PCR kit, a competitive RT-PCR kit, a real-time RT-PCR kit, or a DNA chip kit.

15. A method of treating liver cancer using a vascular endothelial growth factor receptor (VEGFR) inhibitor, the method comprising the steps of:
(a) measuring expression levels of mTOR in samples isolated from liver cancer patients; (b) comparing the expression levels of mTOR with a reference level of mTOR expression, and selecting samples having an mTOR expression level equal to or higher than the reference level; and (c) administering a therapeutically effective amount of the vascular endothelial growth factor receptor (VEGFR) inhibitor to the liver cancer patients showing the mTOR expression level equal to or higher than the reference level.

16. (canceled)
